(19) United States
(12) Patent Application Publication
(10) Pub. No.: US 2025/0135238 A1
(43) Pub. Date: May 1, 2025

(54) SUBCUTANEOUS TRANSCRANIAL FOCUSED ULTRASOUND

(71) Applicant: Blackrock Microsystems, Inc., Salt Lake City, UT (US)

(72) Inventors: Sherman Perry WIEBE, Brussels (BE); Gillian Eden KOEHL, London (GB); Sandeep NEGI, Salt Lake City, UT (US); Robert Kyle FRANKLIN, IV, Farmington, UT (US); Moritz Michael LEBER, Salt Lake City, UT (US)

(21) Appl. No.: 18/924,906

(22) Filed: Oct. 23, 2024

Related U.S. Application Data
(60) Provisional application No. 63/593,921, filed on Oct. 27, 2023.

(51) Int. Cl. A61N 7/00; B06B 1/06

(52) U.S. Cl. CPC A61N 7/00; B06B 1/0622; A61N 2007/0026; A61N 2007/0047; B06B 2201/76

(57) ABSTRACT

Apparatuses, systems, and methods are disclosed for subcutaneous transcranial functional ultrasound technique. An example apparatus includes a substrate and a device configured to be disposed under skin. The device includes one or more transducers disposed on the substrate and provides a pressure wave that propagates a body part under the skin. The pressure wave has a fundamental frequency within a range approximately from 10 Hz to 10 GHz.

### RELATED APPLICATIONS

This application claims priority benefit of U.S. Provisional Patent Application No. 63/593,921, filed Oct. 27, 2023, titled SUBCUTANEOUS TRANSCRANIAL FOCUSED ULTRASOUND, which is incorporated herein by reference in its entirety.

### BACKGROUND

Focused ultrasound (FUS) is a non-invasive brain stimulation technique with neuromodulation of specific brain circuits to treat certain neurological disorders. Ultrasound includes a pressure wave of frequencies above an audible range. As a propagating wave, ultrasound can penetrate biological tissues including a skull. Energy of ultrasound may be concentrated into a small, circumscribed region. A diameter of a stimulated volume is typically several millimeters for applications through a skull, and may reach approximately 100 μm in soft-tissue applications. By applying FUS, cellular activity may be excited or inhibited, depending on specific stimulation parameters. FUS can cause a transient increase in firing rates in motor cortex and in retina with short latency and thus has a direct capability to influence cellular discharge. Transcranial FUS provides non-invasive and reversible approaches for precise (millimeter-level precision) and personalized recording and neuromodulation for neurological treatment or brain computer interfaces. In some examples, FUS neuromodulation in patients with temporal lobe epilepsy has been shown to be safe for relatively high intensities over 5500 mW/cm² doses.

Effectiveness and safety of FUS is limited by the nature of acoustic coupling to the human head. The human skull attenuates ultrasounds by a factor of 4.5 to 64 depending on the individual and skull segment. Hair, acoustic coupling to the head, and entrapped bubbles or air pockets results in severe and highly variable attenuation, which prevents reliable delivery of ultrasound energy to the desired brain targets.

White matter tracts and brain targets (e.g., Brodmann area 25, left dorsolateral prefrontal cortex (DLPFC)) known to be involved in major depression disorders (MDD) have been modulated with transcranial magnetic stimulation (TMS) to provide significant therapeutic relief. FUS stimulation has been shown to treat MDD in a similar way to TMS, by modulating similar brain targets. However, the effects of TMS or FUS treatments may not last long. In the case of FUS treatment for MDD, its effect typically lasts about four to six weeks, and additional repeated therapeutic sessions may be performed every four to six weeks. FUS equipment tends to be large and expensive, thus such equipment may be located at certain clinics. Barriers to scheduling treatment sessions and long commutes to the clinic often result in non-compliance and non-treatment. In addition, most non-invasive FUS treatments are performed in conjunction with computerized tomography (CT) and magnetic resonance imaging (MRI) imaging sessions prior to each treatment, due to small variances in electrode positioning across therapies, to accurately compensate both amplitude and phase attenuation of the ultrasound by the hair, skin and skull. Sensing cortical activity associated with movement intensions, as well as modulating brain regions associated with sensory perception, motor activity, learning and memory, etc., could also be achieved with the present invention.

Recently, the “Relative Through-Transmit” (RTT) technique has been developed. The RTT technique performs direct measurement and compensation for the attenuation and distortion of ultrasound at a given skull and scalp by CT and MRI imaging sessions for the first treatment. However, the RTT technique still causes a patient to visit a clinic for therapy and for recalibration of the non-invasive FUS system on a head of the patient, or results in an error of approximately 1 mm in the x, y, and z directions.

Recent implementations of FUS may also include, for example, a wearable ultrasound phased array patch with flexible complementary metal-oxide semiconductor (CMOS) integrated circuit (IC) chips fabricated through various chip-thinning techniques. The phased array patch is designed for placement on skin outside a human body, such as a head. However, the wearable approach is still ineffective because any FUS device external to a head may need recalibration for each therapy.

The FUS techniques to date have been via external devices and in conjunction with CT and MRI imaging, thereby causing repeated visits to the clinic for the repeated therapy sessions. The repeated sessions impose cost, time and effort on patients, which lead to non-compliance. Moreover, some depressed patients may not have motivation to attend in-clinic repeated treatment sessions over years; thus, the techniques may not be practical to these patients.

### DESCRIPTION OF THE DRAWINGS

FIG. 1 is a schematic illustration of a subcutaneous transcranial FUS (tFUS) system according to some examples.

FIG. 2A is a schematic diagram of a subcutaneous tFUS system according to some examples.

FIG. 2B is a schematic diagram of a subcutaneous tFUS device according to examples described herein.

FIG. 3A is a schematic diagram of a power supply of a subcutaneous tFUS device according to examples described herein.

FIG. 3B is a schematic diagram of a power supply of a subcutaneous tFUS device according to examples described herein.

FIG. 4 is a cross-section of a subcutaneous device according to examples described herein.

FIG. 5 is a schematic illustration of impedance of liquid crystal polymer (LCP) bonded to thermoplastic polyurethane (TPU) under a soak test that may be used in examples of devices described herein.

FIG. 6 is a photograph showing an example of adhesion test results using a 10×10 grid pattern on a borosilicate glass (BSG) substrate that may be used in examples of devices described herein.

FIG. 7 is a schematic illustration of electrical impedance spectroscopy (EIS) of parylene C film that may be used in examples of devices described herein.

FIG. 8 is a schematic illustration of leakage current of four test structures encapsulated with parylene C that may be used in examples of devices described herein.

FIG. 9 is a schematic illustration of impedance and charge storage capacity of an array assembly that may be used in examples of devices described herein.

FIG. 10A is a schematic illustration showing impedance of a subcutaneous device at different time points.

FIG. 10B is a schematic illustration of a cyclic voltammogram of a subcutaneous device at different time points.

FIGS. 11A and 11B show results of relative stability of electrical properties of a subcutaneous device under stimulation according to examples described herein.

FIG. 12 is a schematic illustration showing median impedances for an alumina and parylene bilayer coated electrode array over time of a soak test in phosphate buffered saline (PBS), according to examples described herein.

FIG. 13 is a table listing median impedances for a parylene coated electrode array and alumina and a parylene bilayer coated electrode array for three days of a soak test in PBS, according to examples described herein.

FIG. 14 is a schematic illustration of a relationship between transmitted wireless radio frequency (RF) signal strengths and frequencies monitored as a function of soak time in PBS, according to examples described herein.

### DETAILED DESCRIPTION

Certain details are set forth below to provide a sufficient understanding of embodiments of the disclosure. However, it will be clear to one skilled in the art that embodiments of the disclosure may be practiced without these particular details. Moreover, the particular embodiments of the present disclosure described herein are provided by way of example and should not be used to limit the scope to these particular embodiments. In other instances, well-known materials, components, processes, controller components, software, circuitry, timing diagrams, and/or anatomy have not been described or shown in detail in order to avoid unnecessarily obscuring the embodiments.

A technology for implementing a tFUS system including subcutaneous tFUS devices under skin has been developed. In some examples, encapsulation methods are developed to provide a fully-implanted tFUS device that may perform haemodynamic imaging/sensing or repeated treatments using neuromodulation, such as stimulation and/or suppression of specific brain circuits based on lipid bilayer membrane perforation and/or ion channel modulation. The focused ultrasound device can quantify hemodynamic activity using the doppler effect. The shift in frequency of the emitted wave is due to the motion of the emitter relative to the detector. The tFUS system may cause reversible neuro modulation effect when using power under than 1k W/cm³ and destructive effects when using power greater than 10 kW/cm³. The tFUS system may use a pressure wave with a fundamental frequency within a range approximately from 10 Hz to 10 GHz. The tFUS system may also perform, for example, FUS mediated delivery of gene therapy (AVV) across a blood-brain barrier (BBB) or FUS mediated delivery of cancer therapy across the BBB.

Subcutaneous placement of transducers and sensors enables a portable tFUS device that becomes part of a body. For example, the subcutaneous tFUS device under the skin in a subgaleal space, such as above a skull, may be free from attenuation and distortion of ultrasound by hair and skin. Because of the stability of subcutaneous placement of transducers and sensors, a tFUS system including subcutaneous tFUS devices may provide accurate and effective treatment without repeated recalibrations. Thus, the tFUS system including subcutaneous tFUS device is suitable for continuous and/or chronic operations to provide both monitoring/sensing and neuromodulation treatments. Treatments can be applied to a patient remotely (e.g., outside a treatment room). For example, the treatment may be used in conjunction with a telemedicine video conference call with a care provider of a patient. In some examples, a subcutaneous tFUS system may be combined with a subgaleal ECOG electrode system or a subgaleal electroencephalogram (EEG) electrode system. Subcutaneous device with ultrasound transducers and sensors, light emitters and detectors as well as an EEG electrode may perform combined electrical stimulation, EEG recording, and fNIRS measurements simultaneously for brain activity sensing. The EEG electrode may be used to deliver any combination of transcranial direct current stimulation (tDCS), transcranial alternating current stimulation (tACS), temporal interference (TI) electrical stimulation, intersection short pulse (ISP) stimulation, or other forms of electrical stimulation including charge steering. This device could therefore perform closed loop stimulation treatments.

In some examples, a subcutaneous tFUS system may be combined with a subgaleal ECOG electrode system or a subgaleal electroencephalogram (EEG) electrode system. Subcutaneous device with ultrasound transducers and sensors as well as an EEG electrode may perform combined tFUS neuromodulation, EEG recording, and tFUS sensing measurements simultaneously.

A tFUS device may be fabricated using the micro-electromechanical systems (MEMS) technology. The tFUS device may be insulated using organic or inorganic coatings to prevent moisture ingress. In some examples, a tFUS system may include one or more subcutaneous tFUS devices formed on a substrate including a thin film with a plurality of sheets, such as LCP sheets. Each sheet may have a thickness ranging from 5 μm to 3 mm, thus the subcutaneous tFUS device may fit under the skin. In some examples, a thickness of a subcutaneous tFUS device may be fit within the subgaleal space between the skin and a skull. For example, the thickness of the subcutaneous tFUS device, including a substrate may range from 40 μm to 6 mm. A surface of the substrate may be treated (e.g., encapsulated) with TPU, parylene C (e.g., chlorinated parylene), silicon carbide, atomic layer deposition (ALD) of alumina, epoxy or any combination thereof. For example, a thickness of a TPU layer may range from 5 μm to 3 mm. A thickness of parylene C may range from 3 μm to 50 μm. Silicone thickness may range from 50 μm to 6 mm.

In some embodiments, transducers may be fabricated on the substrate. In some examples, the transducers may be miniaturized curved three-dimensional (3D) transducers. In some examples, the transducers may be formed in single and/or array formats. For example, the transducers may deliver one or more drugs across a BBB. To suit implantable applications, low-voltage, a scandium-doped aluminum nitride (Sc-AlN) MEMS technique may be used to implement piezoelectric micromachined ultrasonic transducers (PMUTs). Curved PMUT membranes may be developed using chip-scale glass-blowing fabrication to obtain high electromechanical coupling coefficients. Curved PMUT arrays use Sc-AlN thin films for piezoelectric material to obtain lead-free and biocompatible implants. Curved 3D PMUTs may reduce a beam width in an elevational direction. Thus, the curved 3D PMUTs may deliver ultrasound energy efficiently to neural targets of interest. Overall, 8×8 PMUT arrays may provide steerable FUS signals at up to 2 cm depth in a brain tissue with a pressure greater than 1 MPa at a focal spot and with a resolution of 0.5 mm or less. The PMUT arrays may be flexible to cover a large region of interest in brain tissue with high resolution for ultrasound stimulation applications.

In some examples, two-dimensional (2D) ultrasound piezoelectric transducer arrays may be fabricated. The transducer arrays may perform beam steering at pressures greater than 500 kPa at a focal target point using a resonant driving frequency of 1.4 MHz. In some examples, each transducer may be biased with a voltage of an arbitrary phase, with a relative offset of individual phases creating a pattern of constructive and destructive interference. Because ultrasound attenuation by a skull may increase with a pressure frequency, a tFUS system may be operated at less than 1.4 MHz. In some examples, multifrequency signals may be used to reduce the focal volume of activation along the axial dimension by a factor of seven. In some examples, a system that is similar to a subcutaneous tFUS device may be utilized in subscalp applications as well.

A subcutaneous tFUS device may include a hermetically sealed ultrasound transducer array in flexible CMOS IC chip for chronic implantation either subcortically, in a subgaleal space, or in an epidural or subdural space above a cortex. The tFUS system may modulate neural circuits to treat neurological disorders such as MDD, anxiety, and/or post-traumatic stress disorders. Targeted continuous deep brain stimulation without brain surgery may treat Parkinson's, essential tremor, and other movement disorders.

In some examples, each subcutaneous tFUS device 102 may include one or more EEG electrodes 122. In some examples, the EEG electrodes 122 may measure electrical activity of neurons underneath the EEG electrodes 122 and provide high temporal resolution, unlike the tFUS hemodynamic sensor. The subcutaneous tFUS device 102 may perform simultaneous collection (e.g., recording) of both tFUS hemodynamic signals and EEG signals and tFUS neuromodulation. In some examples, the EEG signals may be indicative of, for example, short-term motor imagery, whereas the tFUS hemodynamic signals may be indicative of long-term changes, such as cognitive functions or pain. Thus, a user may be able to obtain short-term and long-term brain activity information from the EEG signals and fFUS hemodynamic signals from the subcutaneous tFUS device 102. The EEG signals may also be used to record the impact of the tFUS neuromodulation. In some examples, each of the EEG electrodes 122 may be smaller than each of the detectors 118, and each EEG electrode 122 may be disposed in between adjacent detectors 118, between adjacent transducers 116, or between adjacent transducer 116/detector 118 pairs. Each EEG electrode 122 may detect electrical activity at a proximate spot as the detector 118 detects the tFUS signal.

Treatments using a tFUS system including subcutaneous tFUS devices may be as safe as other non-invasive brain stimulation without risk of seizures. The tFUS system treatments may be performed simultaneously for multiple targets.

The subcutaneous tFUS device may also include an implanted window including a biocompatible material that is transparent to near-infrared wavelengths. The biocompatible material may be a polymer, ceramic, or bioengineered materials such as polymethyl methacrylate (PMMA), yttria-stabilized zirconia (YSZ) with optical clearing agents (OCAs), or any other material which is transparent for ultrasound. In some examples, the implanted window may be a craniofacial implant for repairing a craniofacial defect or replacing other sections of bone in the body. The transparent window may replace at least a portion of a skull, to increase transparency. Thus, the transparent window allows the ultrasound from the device to pass through with less scattering of ultrasound, and increases a signal to noise ratio of the tFUS signal.

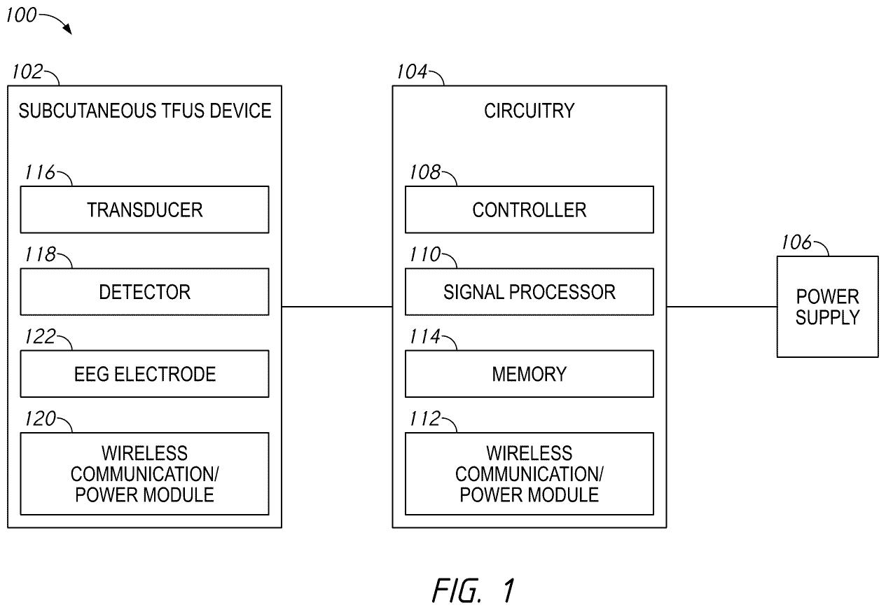

FIG. 1 is a schematic illustration of a subcutaneous tFUS system 100 according to some examples. The subcutaneous tFUS system 100 may include, for example, a processor and one or more memory devices, power supply, and a wireless data transmitter/receiver. In some embodiments, the subcutaneous tFUS system 100 may include one or more subcutaneous tFUS devices 102, circuitry 104, and power supply 106. The power supply 106 may provide power for the circuitry 104. The circuitry 104 may include one or more controllers 108, one or more signal processors 110, memory 114 and a wireless communication/power module 112. In some examples, any of the processor and one or more memory devices, power supply, and the wireless data transmitter/receiver may be provided outside the circuitry 104. Each subcutaneous tFUS device 102 of the one or more subcutaneous tFUS devices 102 may include one or more pairs of transducer 116 and detector 118. In some examples, each pair of transducer 116 and detector 118 may include one or more piezoelectric materials. For example, each pair of transducer 116 and detector 118 may include lead zirconate titanate (PZT). In some examples, the transducers 116 may provide an ultrasound signal. For example, the ultrasound signal may be provided as a pressure wave. In some examples, the pressure wave may have a fundamental frequency in a range from 10 Hz to 10 GHz and may be used for brain stimulation. In some examples, the pairs of transducer 116 and detector 118 may be fabricated in a semiconductor chip, such as a CMOS chip in an integrated manner with detection electronics, such as circuits. In some examples, the integrated CMOS chip may have a thickness that ranges from 5 nm to 50 μm. For example, resolutions of 70 picoseconds may be achieved for time-of-flight imaging using a laser pulse rate of 200 MHz with 80-mW total power.

In some examples, each subcutaneous tFUS device 102 may include a wireless communication/power module 120. In some embodiments, each of the wireless communication/power module 112 and wireless communication/power module 120 may include a low power short distance wireless communication module that may communicate signals, such as control signals and tFUS signals, using, for example, Bluetooth, infra-red, near field communication, etc. Each of the wireless communication/power module 112 and the wireless communication/power module 120 may include a wireless transmitter/receiver. Each of the wireless communication/power module 112 and the wireless communication/power module 120 may include a wireless power charger that may receive power wirelessly. Alternatively or additionally, the subcutaneous tFUS device 102 and the circuitry 104 may include a wired power module that may receive power in a wired manner such as a universal serial bus (USB) or some other chord, etc. The signal processors 110 may process information to provide signals for brain stimulation. The signals for brain stimulation may be tFUS signals or equivalent, or control signals which may cause the transducers 116 to provide tFUS signals. The signals may be transmitted via the wireless communication/power module 112 and the wireless communication/power module 120 to the transducers 116 in the subcutaneous tFUS device 102. The transducers 116 under skin may produce ultrasound signals, such as the tFUS signals.

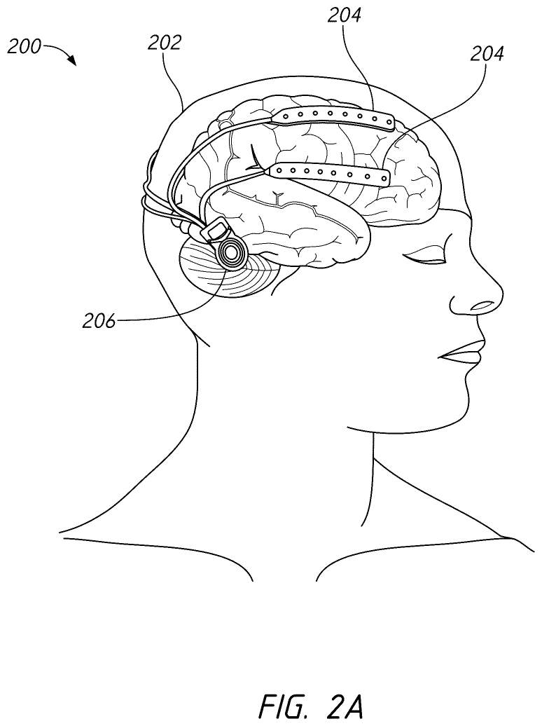

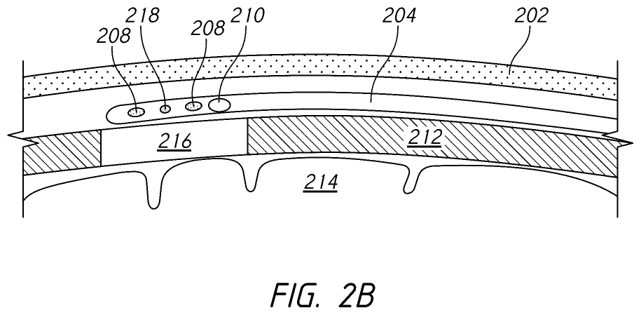

FIG. 2A is a schematic diagram of a subcutaneous tFUS system 200 according to some examples. Generally, examples of systems described herein may include one or more subcutaneous tFUS devices 204. In the example of FIG. 1, the subcutaneous tFUS system 200 includes one or more subcutaneous tFUS devices 204 and circuitry 206 coupled to the subcutaneous tFUS devices 204. In some embodiments, the one or more subcutaneous tFUS devices 204 may be disposed under skin 202. In some examples, the circuitry 206 may also be under the skin 202, or may be disposed on or above the skin 202. In some examples, the subcutaneous tFUS devices 204 in FIG. 2A may be pads. In FIG. 2A, for example, the pads may be linear strips. However, the subcutaneous tFUS devices 204 may be implemented in different shapes suitable for application of ultrasound signals.

FIG. 2B is a schematic diagram of a subcutaneous tFUS device 204 according to examples described herein. In some embodiments, each subcutaneous tFUS device 204 may include one or more transducers 208 and one or more detectors 210. In some examples, each subcutaneous tFUS device 204 may further include one or more EEG electrodes 218. In some examples, each of the EEG electrodes 218 may be smaller than each of the detectors 210, and each EEG electrode 218 may be disposed in between adjacent transducers 208, between adjacent detectors 210, or between adjacent transducer 208/detector 210 pairs. In some examples, the subcutaneous tFUS device 204 may be disposed in a subgaleal space under the skin 202 and on or above a skull 212 protecting a brain 214. In some examples, the transducers 208 and the detectors 210 may be the transducers 116 and the detectors 118 of FIG. 1. In some examples, the subcutaneous tFUS device 204 may include tapering (not shown) on edges that reduces exposure of the subcutaneous tFUS device 204 to outside the skin.

In some embodiments, an implanted window 216 may be disposed on the skull 212. In some examples, the subcutaneous tFUS device 204 may be placed on the skull 212 in a manner that some of the transducers 208 and detectors 210, and/or the EEG electrodes 218 may be placed on or above the implanted window 216. In some examples, the implanted window 216 may be a craniofacial implant for repairing a craniofacial defect or replacing other sections of bone in the body. For example, the implanted window 216 may include one or more transparent biocompatible materials, for example, transparent PMMA plastic, YSZ ceramic with OCAs, or any other transparent biocompatible material suited for safe use in craniofacial reconstruction. While a transparent PMMA craniofacial implant is the preferred embodiment, the prefabricated transparent custom craniofacial implant may include a polymer, metal, bioengineered material, or any combinations thereof for which may also be substantially transparent. In some examples, PMMA, YZS with OCAs, and similar ceramic and polymer materials are transparent to near infrared light signals having a wavelength that ranges from 800 nm to 2500 nm. In some examples, transparent implanted window 216 may replace at least a portion of a skull, to increase transparency. Thus, the transparent window allows the light from the device to pass through with less scattering of ultrasound, and increases a signal to noise ratio of the tFUS signal.

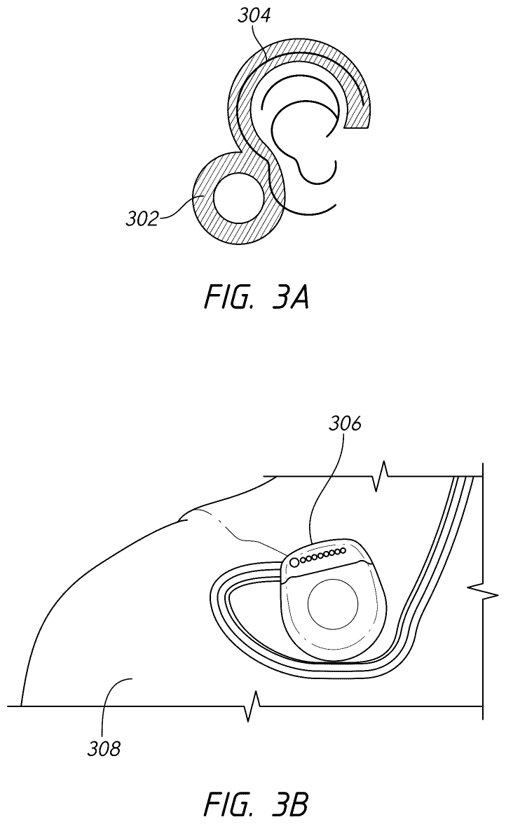

In some examples, a power supply for a subcutaneous tFUS system 200 may be outside the circuitry 206. In some examples, a power supply may be disposed on an ear. FIG. 3A is a schematic diagram of a power supply 302 of a subcutaneous tFUS system 200 according to examples described herein. The power supply 302 may be disposed on or embedded under the skin of an ear 304. The power supply 302 may supply power to the subcutaneous tFUS devices 204 via the circuitry 206.

In some examples, a power supply may be disposed on a chest. FIG. 3B is a schematic diagram of a power supply 306 of a subcutaneous tFUS system 200 according to examples described herein. The power supply 306 may be disposed on or embedded under the skin of a chest 308. The power supply 306 may supply power to the subcutaneous tFUS devices 204 via the circuitry 206.

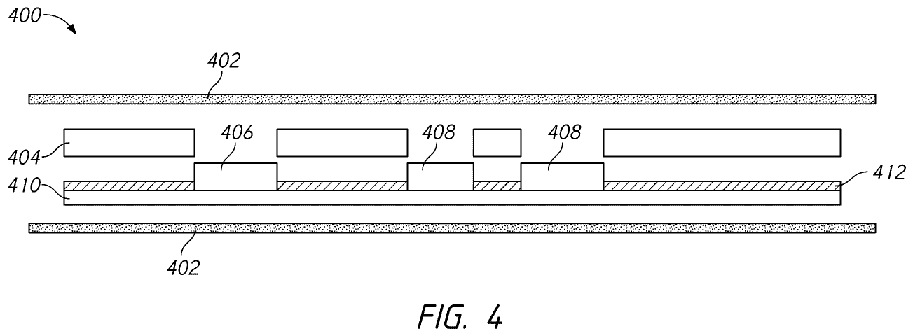

FIG. 4 is a cross-section of a subcutaneous device 400 according to examples described herein. In FIG. 4, the subcutaneous device 400 may be implemented as a semiconductor chip. The subcutaneous device 400 may include top and bottom heating bars 402, stencils 404, one or more transducers 406, and one or more detectors 408 on a substrate 410. A face of the substrate 410 not covered with the transducers 406 and the detectors 408 may be covered with a surface 412 by surface treatment. In some examples, the transducers 406 and the detectors 408, such as the transducers 116 and the detectors 118, may be encapsulated in the substrate 410 including polymer. In order to implant the subcutaneous device 400 in a human body, the subcutaneous device 400 may be manufactured with biocompatible materials that are resilient to chronic implantation in the body. The subcutaneous device 400 may be hermetically sealed for chronic implantation either subgaleally or on the cortex.

In some examples, the substrate 410 may be a polyimide substrate. In some examples, the substrate 410 may be a flexible printed circuit board (PCB) substrate. For example, the substrate 410 may be a flexible liquid crystal polymer thin-film (LCP-TF) substrate including LCP. For example, a substrate including LCP may have longevity and low water permeability compared to a polyimide substrate (e.g., up to 25 times less than polyimide substrates) and reliability and lifetime of an implanted array in the substrate may be extended. In some examples, the LCP-TF substrate may have a thickness that may range from less than 5 μm to 3 mm. In some examples, the substrate 410 may include a TPU layer. TPU has been used in the medical industry due to several properties. TPU has water, fungus, and abrasion resistance. TPU's rubber-like elasticity ensures flame retardancy at varying opacities. TPU polymers having robust mechanical properties, durability, chemical and oil resistance, and biocompatibility are highly desirable for implantable devices. TPU polymers having customizable mechanical properties (e.g., hardness from 72A to 60D Shore durometers) and chemistries (e.g., hydrophilic and hydrophobic) may provide precise control over drug elution and compatibility may be possible. In some examples, the TPU layer may have a thickness in the range of 5 μm to 3 mm. In some examples, the substrate 410 may include silicone. For example, a thickness of silicone may range from 50 μm to 6 mm. In some examples, the substrate 410 may include any combination of LCP, silicone or TPU thereof to reduce water ingress due to chronic implantation.

In some examples, the transducers 406 may provide an ultrasound signal. For example, the ultrasound signal may be provided as a pressure wave. In some examples, the pressure wave may have a fundamental frequency in a range from 10 Hz to 10 GHz, which may be used for brain stimulation. For example, resolutions of 70 picoseconds may be achieved for time-of-flight imaging using a laser pulse rate of 200 MHz with 80-mW total power.

The transducers 406 and the detectors 408 may include one or more piezoelectric materials. For example, the transducers 406 and the detectors 408 may include lead zirconate titanate (PZT). In some examples, the transducers 406 and the detectors 408 may be fabricated in a semiconductor chip, such as a CMOS chip in an integrated manner, such as circuits.

In some examples, the transducers 406 may be miniaturized curved 3D transducers. In some examples, the transducers 406 may be formed in single and/or array formats. For example, the transducers 406 may deliver one or more drugs across a BBB. To suit implantable applications, low-voltage, a scandium-doped aluminum nitride (Sc-AlN) MEMS technique may be used to implement PMUTs. Curved PMUT membranes may be developed using chip-scale glass-blowing fabrication to obtain ultra-high electromechanical coupling coefficients. Curved PMUT arrays may use Sc-AlN thin films for piezoelectric material to obtain lead-free and biocompatible implants. Curved 3D PMUTs may reduce a beam width in an elevational direction. Thus, the curved 3D PMUTs may deliver ultrasound energy efficiently to neural targets of interest. Overall, 8×8 PMUT arrays may provide steerable FUS signals at up to 2 cm depth in a brain tissue with a pressure greater than 1 MPa at a focal spot and with a resolution of 0.5 mm or less. The PMUT arrays may be flexible to fit to a curved shape of a body part, such as a skull, to cover a large region of interest in a brain with high resolution for ultrasound stimulation applications.

In some examples, the transducers 406 may be formed as two-dimensional (2D) ultrasound piezoelectric transducer arrays. The transducers 406 may perform beam steering at pressures greater than 500 kPa at a focal target point using a resonant driving frequency of 1.4 MHz. In some examples, each transducer of the transducers 406 may be biased with a voltage of an arbitrary phase, with a relative offset of individual phases creating a pattern of constructive and destructive interference. Because ultrasound attenuation by a skull may increase with a pressure frequency, a tFUS system may be operated at less than 1.4 MHz. In some examples, multifrequency signals may be used to reduce the focal volume of activation along the axial dimension by a factor of seven. In some examples, a device that is similar to a subcutaneous tFUS device may be utilized in subscalp applications as well.

In some embodiments, metal such as gold, silver or copper may be used as conductive material for wirings (not shown) on the substrate 410. In some examples, to improve stability in vivo chronically, metal such as platinum, iridium, iridium oxide, or any combination thereof may be used as conducting material for device pads (e.g., microcontacts) on the substrate 410. Additionally, gold, iridium oxide and/or platinum-iridium may be used for device pads and leads for biocompatible interaction with the body.

The stencils 404 may be designed to prevent direct stress onto the components. The stencils 404 may cover the substrate 410, except the transducers 406 and the detectors 408. The stencils 404 may be processed with micro-edging around the transducers 406 and the detectors 408. The micro-edging in conjunction with precise alignment during a manufacturing process of the subcutaneous device 400 may reduce an amount of stress to be applied to the components.

Concise explanation of fabrication of a flexible substrate 410 is as follows. In some examples, the transducers 406, such as a transducer array, may be formed as one or more patterns using photolithography on the flexible substrate 410. In some examples, parylene layers may be formed on the substrate 410. Traces may be formed on one of the parylene layers and electroplated, and another parylene layer may be formed to cover the electroplated traces. In some examples, the parylene layer on the electroplated traces may be opened as patterns to expose the electroplated traces. For example, a sheet of piezoelectric material, such as lead zirconate titanate (PZT) or other types of piezoelectric materials [including, but not limited to, piezoelectric crystals such as 0.3Pb (Mg1/3Nb2/3)O3-0.7Pb(Zr0.52Ti0.48)O3/Pb(Zr0.52Ti0.48)O3 (PMN-PT), α-quartz or β-quartz (SiO₂), zinc oxide (ZnO), gallium nitride (GaN), piezoelectric ceramics such as PZT and (Na_{0.5,} K_{0.5})NbO₃ (NKN), aluminum nitride (AlN), (Na, Ca) (Mg, Fe)₃B₃Al₆Si₆(O, OH, F)31 (tourmaline), Ca₃Ga₂Ge₄O₁₄ (CGG), lithium niobate (LiNbO₃), lithium tantalate (LiTaO₃), and piezoelectric polymers such as polyvinylidene fluoride (PVDF), ferroelectric poly (vinylidene fluoride-co-trifluoroethylene) (P(VDF-TrFE)), and poly-L-lactic acid (PLLA)] may be electrically contacted by patterning pads on either side of the substrate 410. Subsequently, pillars are then formed in the PZT sheet by mechanical dicing and are bonded to the substrate 410. On a top surface, sputter deposition of conductive material may be performed to provide a connector circuit to a ground voltage. Thus, a flexible substrate 410 with the transducers 406 and the detectors 408 encapsulated with the parylene layers for insulation may be formed.

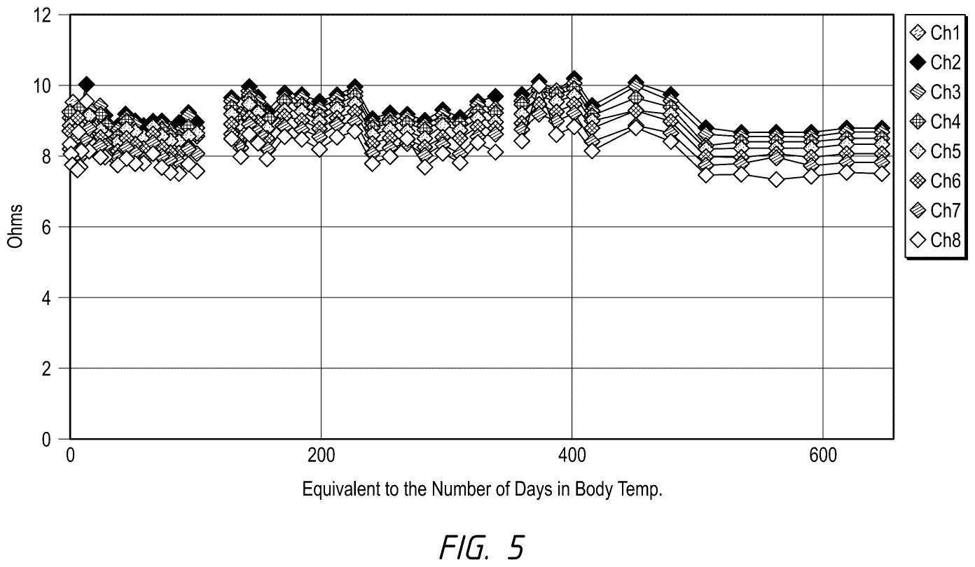

Some components of the flexible substrate 410 including biocompatible materials will be explained in detail referring to FIGS. 5-13. In some examples, the subcutaneous device 400 may be an LCP-TF device. An LCP-TF device has great stability during accelerated aging and in vivo implantation tests. In some examples, an LCP-TF device including two LCP sheets, each having less than 30 μm (e.g., 25 μm) thickness, with wirings in between, may be fused into a single layer at 282° C. under 100 pounds per square inch of force. The LCP-TF device may have resistance to delamination, compared to polyimide device arrays suffering from delamination due to their reliance on inter-layer adhesion. A soak test at 60° C. revealed a lifetime exceeding five years at 37° C. equivalence. In some examples employing rats and non-human primates, implanted LCP-TF device demonstrated chronic signal reliability and stable performance for longer lifetime than polyimide device.

Furthermore, LCP bonding to TPU may provide material properties of both TPU and LCP, ensuring robustness even during accelerated aging conditions. The bonding procedure may include applying heat and pressure to the substrate 410 to melt and blend the two materials together. In some examples, a hydraulic press equipped with heating plates may be used to bond a sandwich structure together. Thorough cleaning, pre-processing, and precise alignment of the silicone, teflon, TPU, LCP device and stainless sheet (SS) sheet may ensure correct bonding profiles and seamless mold release of the LCP and TPU. Once the pressure is released, the entire setup is removed from the press, and a mold is disassembled to retrieve the bonded profiles. The bonded profiles may be cut using a laser marker, followed by removal of excess TPU. Subsequently, the subcutaneous device 400 may be fabricated. A soak test of the LCP bonded to TPU was performed under 37° C. FIG. 5 is a schematic illustration of impedance of the LCP bonded to TPU under a soak test that may be used in examples of devices described herein. In some examples, a subcutaneous device may include a plurality of electrodes. In FIG. 5, an example device including eight electrodes providing eight channels was tested at 37° C., which is normal body temperature. Therefore, the aging of the device was at a normal speed (i.e. 1 day of testing equaled 1 day in the body at room temperature). Impedance values of the eight electrodes (Ch1-Ch8) were relatively stable (around 8Ω) over 400 days. The LCP bonded to TPU demonstrated great impedance stability.

As shown above, the transducers 406 and the detectors 408 may be implemented in a size to provide TD-DOT for subcutaneous (e.g., subgaleal) implantation. Disposing the subcutaneous device 400 between two polyurethane sheets to be blended and melted together to protect a chip, such as the subcutaneous device 400, may be used to protect from fluid ingress. LCP can also be used in conjunction with TPU. The fusing process can be performed using a heated hydraulic press as detailed in the preliminary data shared on fusing LCP and TPU constructs. The TPU encapsulated chip can then be laser cut to the appropriate dimensions.

Surface treatment may be performed on the substrate 410 and the surface 412 may be formed on the substrate 410. The surface 412, including at least one of encapsulation and stencils 404 covering components including the emitters 406 and the detectors 408, may protect the components from any pressing and heating applied to the substrate 410. For example, encapsulation around each component may provide a cushion that may reduce stress onto the component. Encapsulation may use at least one of TPU, parylene C, such as parylene C, silicon carbide, or ALD of alumina that excel in water permeability, mechanical strength, and overall stability. In some examples, the surface treatment may provide an ionic barrier between physiological fluids and components to be insulated. For example, a thickness of a TPU layer may range from 10 μm to 2.5 mm. A thickness of parylene C may range from 3 μm to 50 μm. Silicone thickness may range from 50 μm to 2.5 mm.

Robust parylene C encapsulation methods may result in suitable adhesion and electrical insulation for encapsulation of the subcutaneous device 400. Parylene C having chemical inertness, low dielectric constant (εᵣ=3.15), high resistivity (˜10¹⁵ Ω·cm), and a relatively low water vapor transmission rate of 0.2 g·mm/m²·day has been a reliable coating material for biomedical implantable devices. In some examples, parylene C may be applied by chemical vapor deposition (CVD) at room temperature to form a conformal, pin-hole-free film, without solvents. Moreover, parylene C has ion barrier properties for neural interfaces exposed to physiological fluids.

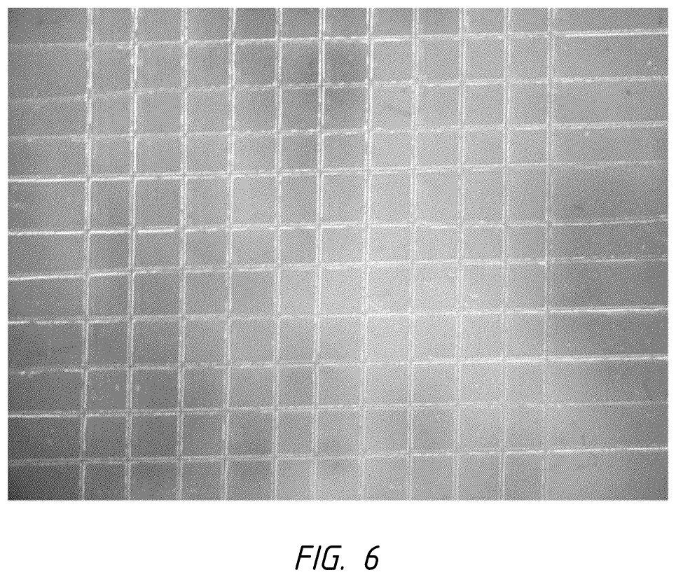

Using an adhesion promoter, such as methacryloxy functional trimethoxy silane (e.g., Silquest A-174 silane), may enhance adhesion of parylene C film. This enhanced adhesion may be observed with both silicon and BSG substrates achieving grade 5B adhesion in a tape adhesion test, whereas substrates without the adhesion promoter exhibited a grade of 0B. FIG. 6 is a photograph showing an example of adhesion test results using a 10×10 grid pattern on a BSG substrate that may be used in examples of devices described herein. All the 3 to 4.5 μm thick parylene C squares made by the cuts remained on the BSG substrate. The pitch of the cuts is 1 mm. Assessment of adhesion results under the ASTM D3359 B standard highlights improvements in adhesion of 3 μm to 4.5 μm thick parylene C layers when using the adhesion promoter. The adhesion promoter effectively mitigates delamination of the encapsulation and ensures bonding.

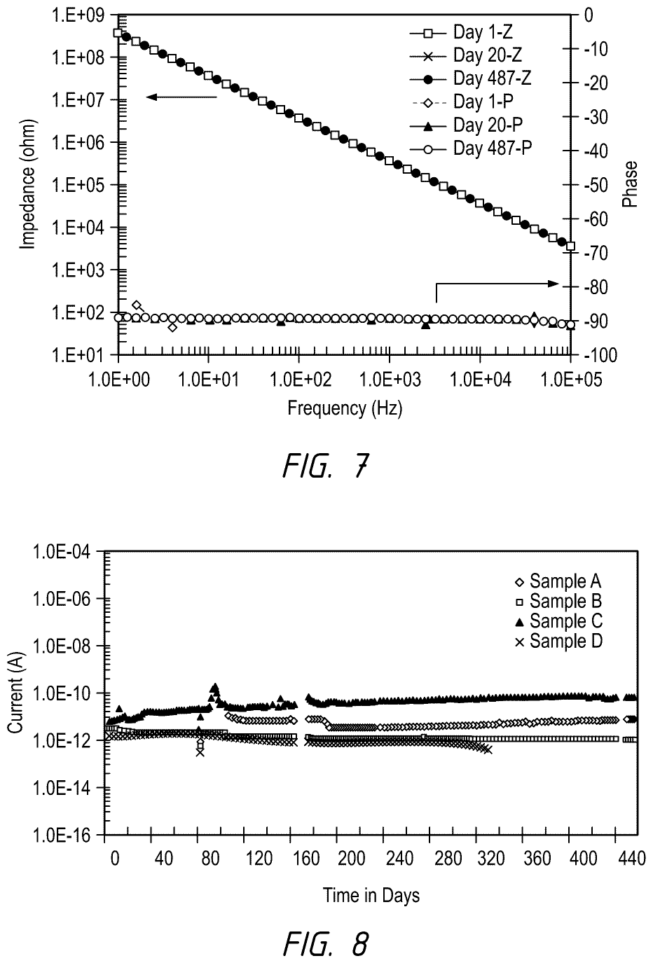

Electrical performance of parylene C layers was tested using EIS and leakage current tests. FIG. 7 is a schematic illustration of EIS of parylene C film that may be used in examples of devices described herein. The parylene C layers may be selected over a 487-day soaking test in 37° C. saline. The impedance was denoted by Z and the phase was denoted by P in FIG. 7. The EIS observed a phase angle of −90° that implies a capacitor behavior, indicating absence of cracks or pinholes in the encapsulation film. The consistency in capacitance across different time points strongly suggests absence of degradation or water absorption within the film.

FIG. 8 is a schematic illustration of leakage current of four test structures encapsulated with parylene C that may be used in examples of devices described herein. In this example, a thickness of parylene C is less than 4.5 μm. Test samples were applied with a 5 V_{dc} bias, and the leakage currents were monitored as a function of time for more than one year. Sample D was removed after 320 days due to failure of an electrical connection. Upon immersion in saline, the leakage currents displayed a slight increase but consistently remained below 10^{\hat{ }-10} A throughout the 450-day testing period. This leakage behavior indicates that the presence of the 5 V_{dc} bias on samples immersed in saline did not compromise the integrity of the parylene C encapsulation.

The EIS and leakage current test results of FIGS. 7 and 8 show that parylene C is a good electrical insulator for neural interface devices and that parylene C may provide insulation at 37 C saline under biased or nonbiased conditions for more than a year.

In some examples, the surface treatment including, for example, ALD of alumina or silicon carbide, may be used to improve electrical properties and stability of the subcutaneous device 400 of FIG. 4.

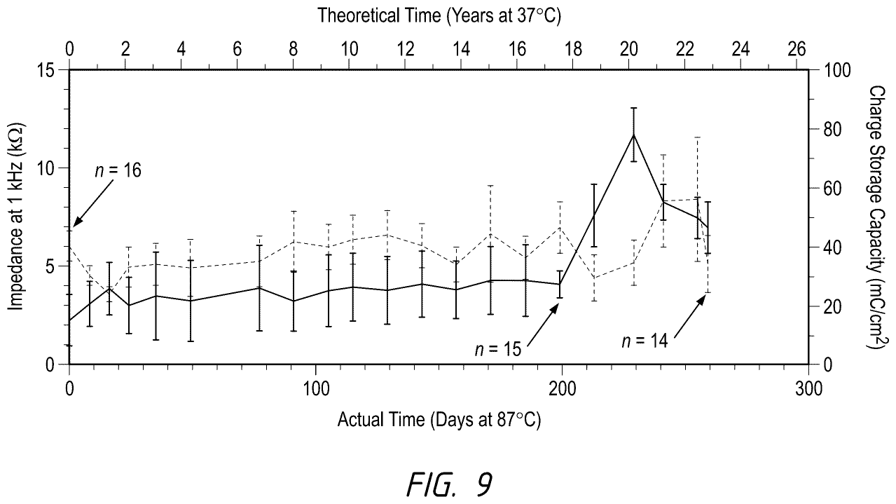

In some examples, silicon carbide may be used to enhance the electrical properties and stability at tissue interfaces of subgaleal devices. A high-channel count microelectrode array, such as the Utah electrode array (UEA), may be coated with silicon carbide, and its electrical properties and stability may be tested over time. The array assembly was soaked in PBS at 87° C., and an impedance at 1 kHz and charge storage capacity (CSC) were measured. Numbers of samples are 15 and 14, respectively. FIG. 9 is a schematic illustration of the impedance and CSC of the array assembly that may be used in examples of devices described herein. FIG. 9 shows stability of electrical properties of the array coated with silicon carbide over 250 days at 87° C., theoretically equivalent to 22 years at 37° C.

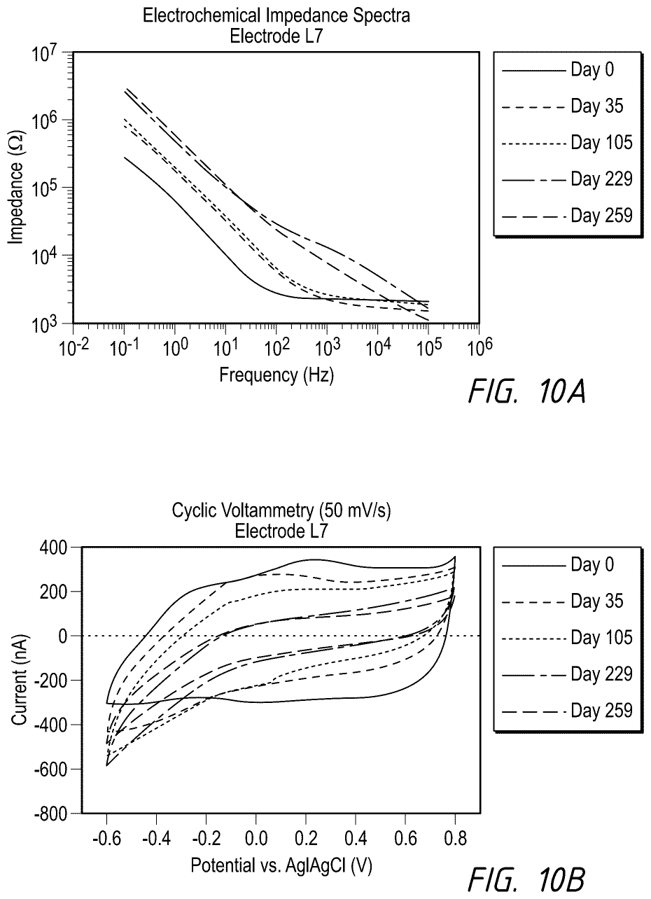

Cyclic voltammetry and impedance at different timepoints of a subcutaneous device going through aging are obtained. FIG. 10A is a schematic illustration showing the impedance of a subcutaneous device at different time points. FIG. 10B is a schematic illustration of a cyclic voltammogram of a subcutaneous device at different time points.

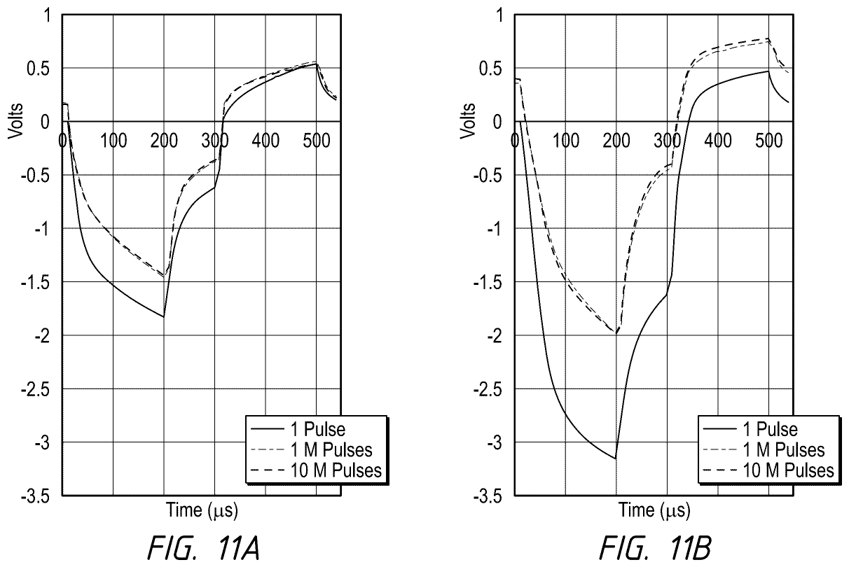

The stability of the silicon carbide coating during stimulation was investigated since it may sometimes drive unwanted chemical and physical reactions which damage the devices. FIGS. 11A and 11B show results of relative stability of electrical properties of a subcutaneous electrode under stimulation according to examples described herein. Electrode sites on two different UEAs (A and B) were subjected to 10 million stimulation pulses and voltage transients were recorded. The amplitude of the voltage transient decreased within the first 1 million pulses, then remained stable for 10 million pulses.

Mechanical stability of the silicon carbide was investigated through an electrode insertion test and a wire bend test. The insertion of a UEA into 1% agarose-PBS gel (model cortex material) resulted in no damage to devices tested. The devices that were damaged prior to insertion retained their structure and no additional damage was observed.

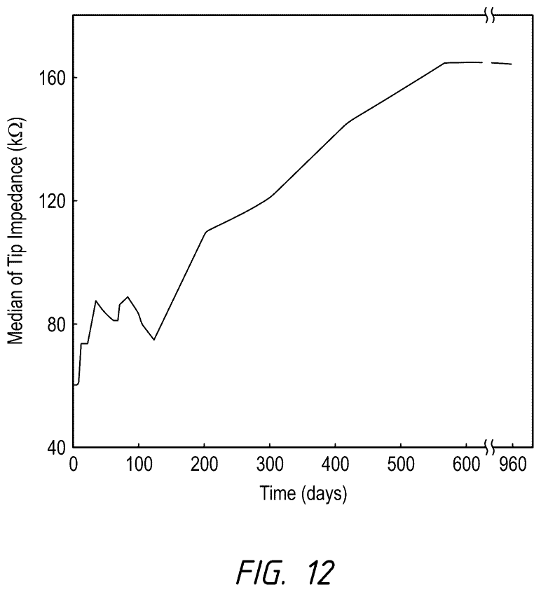

In some examples, ALD of alumina may be used to enhance the electrical properties and stability at the tissue interface in subgaleal devices. For example, ALD of alumina may also be deposited prior to parylene C encapsulation in order to improve the impedance, signal stability and strength, and current draw long term reliability. FIG. 12 is a schematic illustration showing median impedances for an alumina and parylene bilayer coated electrode array over time of a soak test in PBS at 37° C., according to examples described herein. (“Long-term reliability of Al2O3 and parylene C bilayer encapsulated Utah electrode array based neural interfaces for chronic implantation”, J. Neural Eng. 11 (2014)). Different from the trend of continuous drop in impedance for parylene coated arrays, median impedances of alumina and-parylene bilayer coated wired arrays increased from 60k Ω to 160kΩ after 960 equivalent days of soak testing at 37° C. For bilayer coated arrays, the loss of iridium oxide and etching of silicon in PBS solution (leading to impedance increase) dominates over the slow bilayer encapsulation degradation (resulting in decreased impedance).

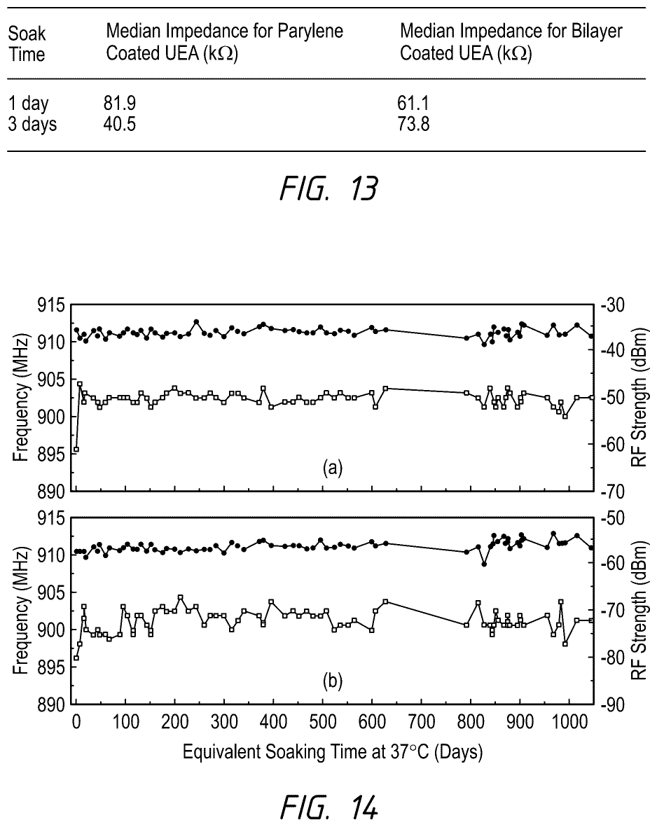

In an accelerated lifetime test, wired, wireless UEAs and active arrays were soaked in PBS at 57° C. FIG. 13 is a table listing median impedances for parylene coated UEA and alumina and parylene bilayer coated UEA for three days of a soak test in PBS, according to examples described herein. Id.

FIG. 14 is a schematic illustration including a relationship between transmitted wireless RF signal strengths and frequencies monitored as a function of soak time in PBS according to examples described herein. In (a) of FIG. 14, signals are extracted from customized wireless unit, and in (b) of FIG. 14, signals were measured using a spectrum analyzer. In both measurement methods, the RF signal strengths and corresponding frequencies stayed relatively stable during 1044 days of equivalent soak time at 37° C. Bilayer coated wireless UEAs incorporated with active electronics had stable power-up frequencies of ˜910 MHz and constant RF signal strengths of ˜−50 dBm (measured by hand receiver) over 1044 equivalent days of soak testing at 37° C., showing the slow water ingress and excellent insulation performance of the bilayer encapsulation. The current draw of active arrays was constant at ˜3 mA with a power supply of V_{dd} at 1.5V and Vₛₛ at −1.5V during 228 equivalent days of soak testing at 37° C. The low and constant current draw is a reliable indication of good protection of the device by the encapsulation. Based on the coating performance on neural interfaces, the bilayer encapsulation may be used for many other chronic biomedical implantable devices to increase the lifetime of the devices.

The subcutaneous tFUS techniques and methods to manufacture subcutaneous tFUS devices described herein may be used for chronic treatment of the brain.

Examples provided herein of both the design of subcutaneous tFUS devices and the clinical applications are not the limit of the uses of the subcutaneous tFUS devices in the subgaleal zones. Many configurations of FUS systems including subcutaneous tFUS devices exist, as well as applications that would benefit from the use of the technology described herein.

It is to be appreciated that any one of the above embodiments or processes may be combined with one or more other embodiments and/or processes or be separated and/or performed amongst separate devices or device portions in accordance with the present systems, devices and methods.

Finally, the above discussion is intended to be merely illustrative of the present devices, apparatuses, systems, and methods and should not be construed as limiting the appended claims to any particular embodiment or group of embodiments. Thus, while the present disclosure has been described in particular detail with reference to exemplary embodiments, it should also be appreciated that numerous modifications and alternative embodiments may be practiced without departing from the broader and intended spirit and scope of the present disclosure as set forth in the claims that follow. Accordingly, the specification and drawings are to be regarded in an illustrative manner and are not intended to limit the scope of the appended claims.

What is claimed is:

1. An apparatus comprising:
a substrate and a device configured to be disposed under skin, the device comprising:
one or more transducers disposed on the substrate and configured to provide a pressure wave, the pressure wave being configured to propagate a body part under the skin,
wherein the pressure wave comprises a fundamental frequency in a range approximately from 20 kHz to 10 GHz.

2. The apparatus of claim 1, wherein a thickness of the apparatus ranges from 40 μm to 6 mm.

3. The apparatus of claim 1, further comprising tapering on edges of the device, the tapering configured to reduce the visibility of the device contour from outside the skin.

4. The apparatus of claim 1, further comprising a surface on the substrate, wherein the surface includes at least one of TPU, parylene C, silicon carbide or ALD of alumina, epoxy, or other organic or inorganic coatings to prevent moisture ingress, or any combination thereof.

5. The apparatus of claim 1, wherein the substrate comprises at least one of the LCP, silicone or TPU layers or any combination thereof.

6. The apparatus of claim 5, comprising an LCP layer comprising a plurality of LCP sheets.

7. The apparatus of claim 6, wherein each LCP sheet of the plurality of LCP sheets has a thickness ranging from 5 μm to 3 mm.

8. The apparatus of claim 1, wherein the device is sandwiched between two or more LCP sheets.

9. A method comprising:
forming a substrate;
performing surface treatment on the substrate, the surface treatment including encapsulating a surface of the substrate; and
forming one or more transducers on the substrate.

10. The method of claim 9, wherein the transducers comprise PZT.

11. The method of claim 9, wherein forming the substrate includes bonding LCP to TPU.

12. The method of claim 9, wherein performing the surface treatment includes encapsulating the surface of the substrate with at least one of TPU, parylene C, silicon carbide, ALD of alumina or epoxy.

13. The method of claim 12, wherein encapsulating the surface of the substrate with parylene C comprises using an adhesion promoter.

14. A system comprising:
circuitry that includes a processor configured to provide signals for brain stimulation; and
a subcutaneous device configured to be disposed under skin, the subcutaneous device including one or more transducers disposed on the substrate and configured to provide one or more ultrasound signals as a pressure wave responsive to the signals for brain stimulation from the circuitry.

15. The system of claim 14, wherein the pressure wave comprises a fundamental frequency in a range approximately from 10 Hz to 10 GHz, the pressure wave being configured to propagate a body part under the skin.

16. The system of claim 14, wherein the subcutaneous device is a subgaleal device configured to be disposed on or above a skull of a head,
wherein the one or more ultrasound signals are configured to propagate the skull and a brain of the head.

17. The system of claim 14, wherein the subcutaneous device further comprises one or more EEG electrodes configured to obtain an EEG signal.

18. The system of claim 17, wherein each EEG electrode of the one or more EEG electrodes is disposed between adjacent transducers.

19. The system of claim 16, further comprising a window replacing at least a portion of the skull, the window having a higher transparency than the skull, configured to allow the ultrasound signals from the transducer to pass through with less scattering of the ultrasound signals than the skull.
